(19) United States
(12) Patent Application Publication
(10) Pub. No.: US 2019/0247013 A1
(43) Pub. Date: Aug. 15, 2019

(54) DETECTION OF MICROCALCIFICATIONS IN ANATOMY USING QUANTITATIVE TRANSMISSION ULTRASOUND TOMOGRAPHY

(71) Applicant: QT Ultrasound LLC, Novato, CA (US)

(72) Inventors: Bilal Hameed MALIK, Novato, CA (US); Mark Wayne LENOX, College Station, TX (US)

(21) Appl. No.: 16/276,531

(22) Filed: Feb. 14, 2019

Related U.S. Application Data
(60) Provisional application No. 62/630,420, filed on Feb. 14, 2018.

(51) Int. Cl. A61B 8/08; A61B 8/14; G06T 7/00

(52) U.S. Cl. CPC A61B 8/0825; A61B 8/5207; A61B 8/5269; A61B 8/085; A61B 8/14; G06T 2207/30068; G06T 2207/20036; G06T 2207/10132; G06T 2207/20032; G06T 2207/20182; G06T 7/0014

(57) ABSTRACT

Microcalcifications can be detected using quantitative ultrasound tomography. Refraction-corrected reflection images generated from a quantitative ultrasound system can be thresholded, and morphologically analyzed to isolate voxels corresponding to microcalcifications.

### CROSS-REFERENCE TO RELATED APPLICATION

This application claims the benefit of U.S. Provisional Application Ser. No. 62/630,420, filed Feb. 14, 2018.

### BACKGROUND

In breast cancer detection, one possible early indicator for breast cancer or precancerous changes to breast tissue involves certain types of breast calcifications. Breast calcifications are small calcium deposits that can develop in a woman's breast tissue (and may even line blood vessel walls). Calcifications are categorized by form, size, density, and distribution. Many microcalcifications are benign; however, certain shapes and distributions can indicate possible cancer or precancerous cells.

For example, suspicious microcalcifications can be those that are clustered together, exhibit a branching or linear pattern, have variable density of distribution, or are distributed in a segmental or haphazard way. Other features that may be suspicious include the form of the microcalcifications that are either round, oval, punctuate, or amorphous.

A challenge with using microcalcifications as a biomarker for breast cancer detection or screening is that microcalcifications do not generate any special symptoms and are not something that can be felt during a routine breast exam.

Accordingly, much effort has been taken to support identification of microcalcifications using X-ray imaging, such as carried out in mammography. However, these efforts are not applicable to imaging modalities such as ultrasound due, for example, to available resolution of the image. Indeed, while conventional handheld ultrasound (HHUS) has been a mainstay of diagnostic breast imaging, the ability of HHUS for evaluation of breast microcalcifications is somewhat limited and the results to date do not support the clinical use of HHUS for detection of microcalcifications.

### BRIEF SUMMARY

Detection of microcalcifications in anatomy using quantitative transmission ultrasound tomography is described.

A method of detecting microcalcifications includes receiving refraction-corrected reflection images of an anatomy generated from a quantitative transmission ultrasound system; performing a thresholding to, for example, remove bright intensity points; removing voxels corresponding to skin from the image; and performing a morphology analysis. In some cases, texture analysis can be applied. In some cases, the method further includes a step to remove outlier voxels. The outlier voxels may be removed by applying image filters, such as median filter, or by application image de-noising methods. Further optimization and processing such as performing image deconvolution and/or applying a varying log compression to intensity values can be applied.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates a topography of a Quantitative Transmission Ultrasound (QTUS) system.

FIG. 2 illustrates a system environment.

FIG. 3 illustrates a process flow diagram of a process that can be carried out by an acquisition control system.

FIG. 4A illustrates a method of microcalcification detection.

FIG. 4B shows images corresponding to the operations shown in FIG. 4A.

FIG. 5A illustrates a method of microcalcification detection.

FIG. 5B shows images corresponding to the operations shown in FIG. 5A.

FIG. 6 illustrates a method of microcalcification detection incorporating image deconvolution.

FIG. 7 illustrates a method of microcalcification detection incorporating variable log compression.

FIG. 8 shows an example computing system through which microcalcification detection may be carried out.

FIG. 9A shows an agar phantom (liquid phase) with calcium particles to show a comparison with FIG. 9B.

FIG. 9B shows a reflection image of the agar phantom.

FIGS. 10A-10C show formation of the agar phantom from the liquid phase agar phantom of FIG. 9A.

FIGS. 11A and 11B show an X-ray mammography image and QT reflection image, respectively, of an agar phantom with calcium particles.

FIGS. 12A and 12B show an X-ray mammography image and QT reflection image, respectively, of a cadaver breast tissue.

### DETAILED DISCLOSURE

Detection of microcalcifications in anatomy using quantitative transmission ultrasound tomography is described. Refraction-corrected reflection images (e.g., reflection images corrected by using speed-of-sound data) can be processed to extract microcalcifications from background or other anatomy.

Quantitative Transmission Ultrasound (QTUS) performs both reflection and transmission ultrasound methods to gather data. The reflection portion directs pulses of sound wave energy into tissues and receives the reflected energy from those pulses—hence it is referred to as “reflection ultrasound.” Detection of the sound pulse energies on the opposite side of a tissue after it has passed through the tissue is referred to as “transmission ultrasound.” QTUS enables evaluation of tissue in clinical ultrasound by offering high spatial and contrast resolution, with absolute spatial registration (no image warping or stretching) quantitative imaging.

In particular, QTUS uses inverse scatter technology providing transmission information (speed of sound and attenuation) mapping of breast tissue. The speed of sound map, which is essentially related to a map of refractive index values, is then used for refraction correction in the reflection image. Applications describing techniques for inverse scattering include U.S. Pat. Nos. 4,662,222; 5,339,282; 6,005,916; 5,588,032; 6,587,540; 6,636,584; 7,570,742; 7,684,846; 7,699,713; 7,771,360; 7,841,982; 8,246,543; and 8,366,617, which are hereby incorporated by reference in their entirety—except for that which is inconsistent with the apparatus and techniques described herein. The techniques described in “Three-dimensional nonlinear inverse scattering: Quantitative transmission algorithms, refraction corrected reflection, scanner design and clinical results,” Wiskin et al., Proceedings of Meetings on Acoustics, Vol. 19, 075001 (2013), are hereby incorporated by reference in their entirety.

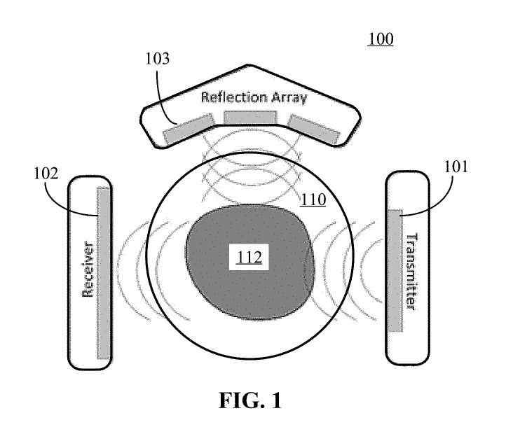

For example, FIG. 1 illustrates a topography of a QTUS system. Referring to FIG. 1, the imaging portion of the QTUS system 100 can include a transmitter 101, receiver 102, and transducer 103. A receptacle 110 is provided to present a water (or other liquid or gel) bath in which a patient may rest at least the region of interest (e.g., the part 112 being imaged). Because the motion artifacts associated with patient movement can affect the image quality, mechanisms can be provided to facilitate retention and positioning of the breast or other body part. For example, an adhesive pad with a magnet can be placed near the nipple region of the breast and docked to a magnetized retention rod that gently holds the breast in a consistent position during the scan. As another example, a membrane over the bath between the breast and the liquid is used to hold the breast (and allow for alternative liquids in the bath).

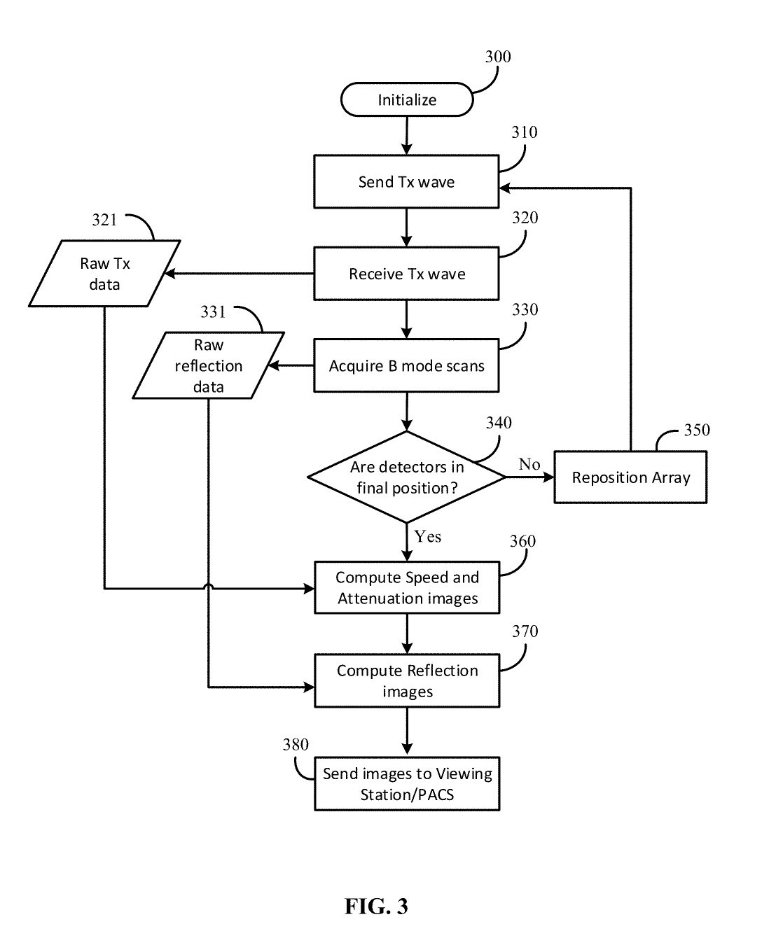

The transmitter 101 and a receiver 102 are provided on opposite sides to enable the performing of transmission ultrasound. The transmitter 101 and the receiver 102 may be in the form of an array of transmitters and receivers. The transmitter array can emit broad-band plane pulses (e.g., 0.3-2 MHz) while the receiver array includes elements that digitize the time signal. A set of reflection transducers 103 are also included to perform reflection measurements. The reflection transducers 103 can include transducers of varying focal lengths, providing a large depth of focus when combined. In the illustration, three transducers are provided. The reflection imaging provides images that represent propensity for reflection information (impedance mismatch) spatially. The reflection images are refraction-corrected and attenuation-calibrated using the speed of sound and attenuation information acquired from the transmission data, for example, as described in more detail with respect to FIG. 3.

360° of data can be obtained through rotation of the system. The system (particularly arms containing the transmitter 101 and the receiver 102) may rotate 360° to acquire measurements from effectively all the angles (e.g., data sufficient to provide a 360° view even if not taken at every angle between 0° and 360°) and collect tomographic views of ultrasound wave data. The reflection transducer data can be collected with one or more horizontal reflection transducers 103 that acquire data in steps or continuously as they rotate 360° along with the transmitter 101 and receiver 102.

In a specific implementation, the system rotates around the patient while both transmission and reflection information are captured. It is not necessary to acquire an entire 360° scan; images can be reconstructed with limited information. For example, a patient can lie prone with their breast pendent in a controlled temperature water bath (e.g., 31° C.) within the field of view of the transmitter 101, receiver 102, and transducer 103 as the transmitter 101, receiver 102, and transducer 103 rotate 360° around the patient. Then, in one example case 180 projections of ultrasound wave data may be obtained. In another example case, 200 to up to 360 projections of the ultrasound wave data may be obtained.

Other detector configurations may be used. For example, additional detectors in a continuous or discontinuous ring or polygon configurations may be used. Of course, any configuration selected will have tradeoffs in speed and cost. In addition, in some cases, reflection arrays (the transducers for the reflection measurements) can do double-duty and perform independent transmission and receiver functions as well as reflection measurements.

As explained above (and in more detail with respect to FIG. 3), the acquired reflection images are spatially compounded and corrected for refraction using the corresponding speed of sound information. The spatial compounding results in significant reduction of image speckle while maintaining the high-resolution nature of the images similar to that of traditional B-mode ultrasound. The end result of each scan may be a 3D volume of essentially three different modalities: speed of sound, attenuation, and reflection. Each of these 3D volume images may be consist of voxels chosen from a range of sizes. For example, in one embodiment a voxel may have dimensions of 400 μm×400 μm×1 mm.

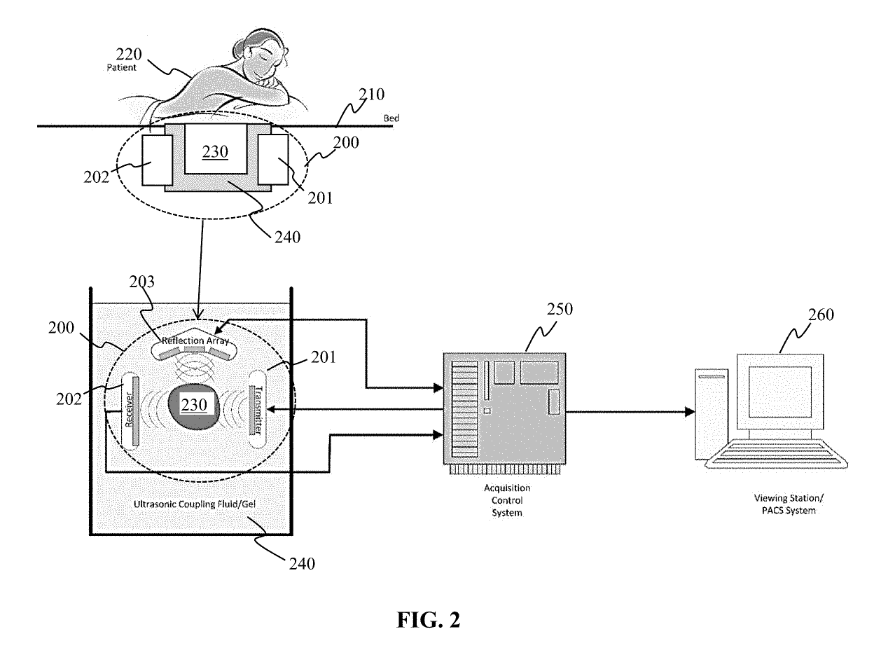

FIG. 2 illustrates a system environment. Referring to FIG. 2, active components (e.g., the imaging components, or transducers, of a QTUS system 200), such as a transmitter 201, receiver 202, and reflection array 203, can be disposed around a receptacle 230 beneath a bed 210 on which a patient 220 can lie. The patient 220 can be scanned in the prone position, resulting in a comfortable procedure. Other configurations are also possible for the apparatus on which the patient is positioned.

The active components (transducers of QTUS system 200) are arranged so that data may be obtained 360° around the receptacle 230 in the bed 210 (via any suitable configuration; and are coupled to the patient with an ultrasonic coupling medium 240 (fluid or gel), at least some of which is disposed in the receptacle 230. An acquisition control system 250 operates the various active components (e.g., the transducers) and can control their physical motion (when system 200 is arranged in a rotating configuration).

The acquisition control system 250 can automate a scan in response to a start signal from an operator. This automated acquisition process does not require operator interaction during the scanning procedure. Once the scan is complete, the acquisition control system 250 (or other computing system having access to the data) can compute the reflection, speed of sound, and attenuation results from the collected data. The acquisition protocol enables temporal comparisons of 3D data sets; and these data sets can be compared in the same plane and orientation as those acquired with other 3D modalities, such as magnetic resonance imaging (MRI). The acquisition control system 250 can transmit the results to a viewing station 260 and/or a picture archival and communication system (PACS). Thus, images can be automatically acquired, stored for processing, and available for physician review and interpretation at the review workstation 260.

FIG. 3 illustrates a process flow diagram of a process that can be carried out by an acquisition control system. In response to receiving an indication to initiate automated scanning (e.g., from an operator), an acquisition control system, such as system 250, can initialize (300) and send a transmission wave from a specified angle about a patient (310), for example from one or more transmitters (such as transmitter 201). As the receiver(s) 202 sense the signal transmitting through the patient (320), raw transmission data 321 is captured. Then, spatially compounded extended depth of focus B mode scans, for example using transceivers 203, are acquired (330) to obtain raw reflection data 331. Of course, in some cases, the B mode scans may be performed before the transmission ones. Additionally, in some embodiments, reflection transducers may have different focal lengths to extend the overall depth of focus within the imaging volume.

The acquisition control system determines whether the detectors are in the final position (340). For a rotating system, the acquisition control system can communicate with a motor control of the platform on which the active components are provided so that a current and/or next position of the platform is known and able to be actuated. For a fixed system, the acquisition control system determines the selection of the active arrays according to an activation program. Accordingly, the “detection” of final position may be based on information provided by the motor control, position sensors, and/or a position program (e.g., using counter to determine whether appropriate number of scans have been carried out or following a predetermined pattern for activating transceivers). If the detectors are not in final position, the acquisition control system causes the array to be repositioned (350), for example, by causing the platform to rotate or by selecting an appropriate array of transceivers of a fixed platform configuration. After the array is repositioned, the transmission wave is sent (310) and received (320) so that the raw transmission data 321 is collected and the B mode scans can be acquired (330) for raw reflection data 531. This repeats until the detectors are determined to be in the final position.

Once all the data is collected (and the detectors completed the final position), speed of sound images, attenuation images, and reflection images can be computed (360, 370). Reflection images may be corrected for refraction with the aid of the speed of sound images (as part of operation 370). In some cases, both the original uncorrected reflection images and the refraction corrected reflection images may be available and sent to a viewing station and/or PACS (e.g., systems 260 of FIG. 2) (380). The computations may be carried out according to methods described, for example, in U.S. Pat. Nos. 5,588,032; 6,005,916; 6,587,540; 6,636,584; 7,570,742; 7,684,846; 7,841,982; and 8,246,543, each of which are incorporated by reference in their entirety except for anything inconsistent with the subject specification. 3D images can be created since the information content of the data is back-projected into the image space as a 3D volume.

Advantageously, information on microcalcifications can be extracted from the refraction-corrected reflection images. The refraction-corrected images from a QT Ultrasound system are specifically shown in the examples provided herein. However, the techniques described herein for microcalcification detection are suitable for any set of reflection images with sufficient resolution. Microcalcifications can be extracted from background and other anatomy of the reflection images by performing a thresholding process and a morphology analysis.

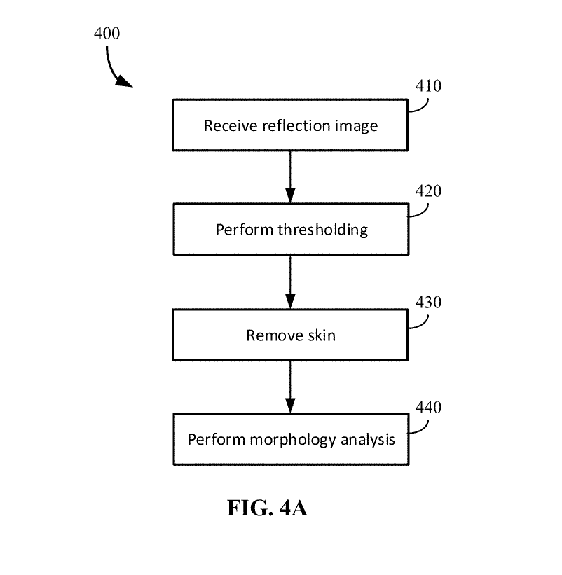

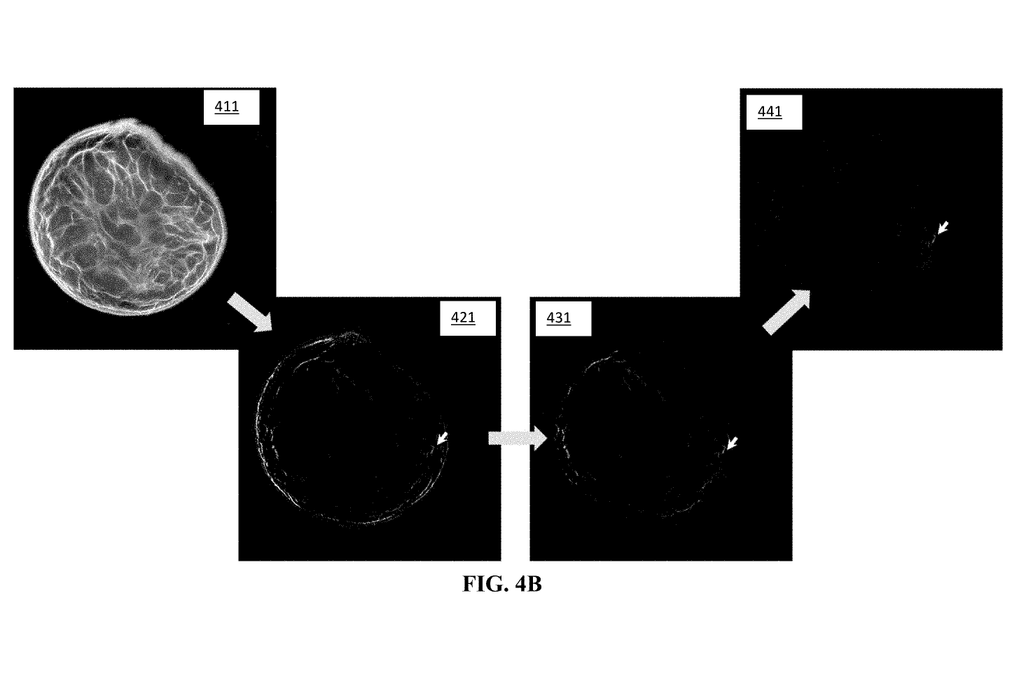

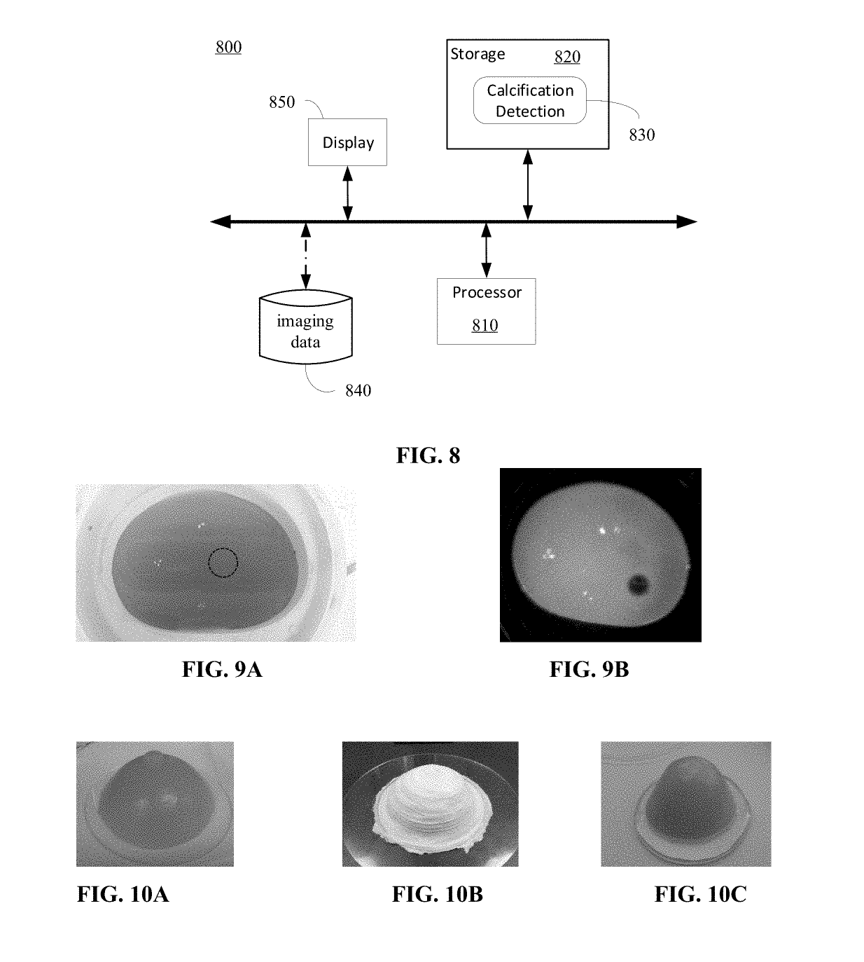

FIG. 4A illustrates a method of microcalcification detection; and FIG. 4B shows images corresponding to the operations shown in FIG. 4A. Referring to FIGS. 4A and 4B, process 400 can begin with receiving refraction-corrected reflection images (410). For example, an image 411 can be received for processing by a computing system such as system 800 described with respect to FIG. 8. A thresholding process (420) can be applied to the image 411. The thresholding process can create a binary image based on the intensity value of each pixel in the image. In some cases, other image segmentation algorithms may be used. Image 421 illustrates an example of an image that may result from thresholding image 411. The arrows (in images 421, 431, and 441) indicate the area to be isolated.

Skin (i.e., pixels/voxels identified as having a reflection value indicative of being skin) is removed (430) from the reflection image. Skin voxel removal can be carried out such as described in application Ser. No. 14/597,707, where the convex nature of the breast can be used to identify and remove voxels considered to be associated with skin. Skin tissue voxels can be identified as high speed voxels that are on a boundary of the breast voxels and exterior voxels. Image 421 with skin removed is shown as image 431. In addition to skin, ligaments and other connective tissue can have high reflection values, which can interfere with identifying microcalcifications. Accordingly, morphology analysis is performed (440) to extract the voxels likely to correspond to microcalcifications from the other high reflective voxels, for example as shown in image 441. The morphology analysis is a three-dimensional analysis but can be carried out for any arbitrary 2D plane. In some cases, texture analysis can be applied.

The morphology analysis can include identifying voxels having an intensity higher than a threshold intensity as satisfying an intensity criterion; and determining whether, for any arbitrary plane in the imaging data, there exists a set of adjacent voxels that satisfy the intensity criterion and a size criterion. An example morphology analysis includes looking for a set of adjacent voxels within a size criterion. The size criterion may be a row (or column) of voxels that is 5 to 20 voxels long. Of course, the specific number of voxels for the size criterion can depend on resolution. The morphology analysis can be carried out in any arbitrary direction.

It should be understood that operations 420, 430, and 440 may be performed in a variety of orders (e.g., skin removal 430, then thresholding 420). In addition, in some cases, some of the processes may be performed in parallel.

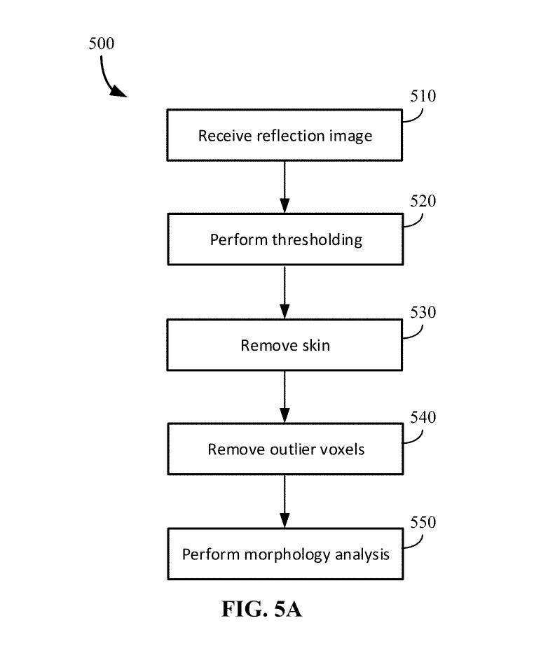

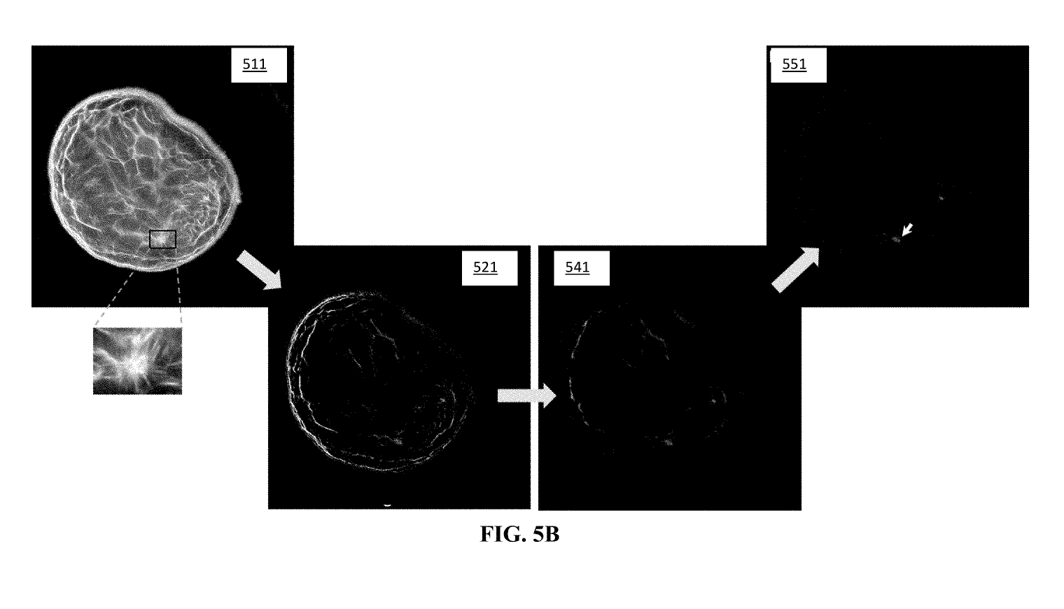

FIG. 5A illustrates a method of microcalcification detection; and FIG. 5B shows images corresponding to the operations shown in FIG. 5A. Process 500 can be similar to that described with respect to process 400, but further includes a step to remove outlier voxels. In particular, referring to FIGS. 5A and 5B, process 500 can include receiving (510) a reflection image 511; performing a thresholding process (520) to generate a thresholded image 521; and removing skin voxels (530). In addition to removing skin voxels, outlier voxels can be removed (540). The outlier voxels may be removed by applying image filters, such as median filter, or by application image de-noising methods. One approach to removing outlier voxels is to compare a current voxel to its adjacent voxel; and if the difference is larger than a threshold, the higher value (higher intensity) voxel is removed as noise. This may be accomplished using, for example, a median filter, or an image de-noising method. A resulting image of the skin voxel removal and outlier voxel removal is image 541 of FIG. 5B. A morphology analysis (550) can be performed to extract the microcalcification, such as shown in image 551 (and identified by the arrow in image 551).

As with method 400, it should be understood that operations 520, 530, 540, and 550 may be performed in a variety of orders. In addition, in some cases, some of the processes may be performed in parallel.

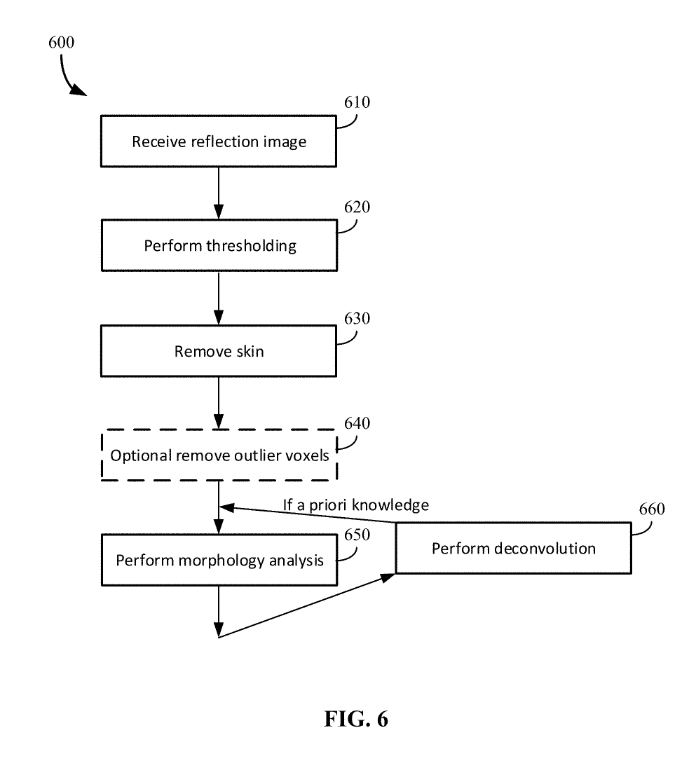

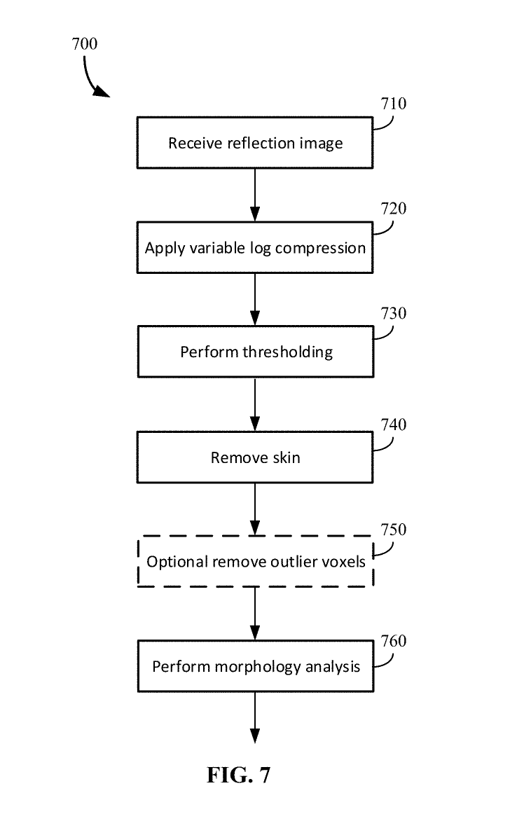

A number of optimizations for the detection of microcalcification may be performed alone or in combination with each other. FIG. 6 illustrates a method of microcalcification detection incorporating image deconvolution; and FIG. 7 illustrates a method of microcalcification detection incorporating variable log compression. Referring to FIG. 6, since an imaging system can be considered a transfer function (i.e., instrument response) and an object can be defined as a function, the final image of the object can be considered a convolution of the object function and the transfer function. Given some apriori knowledge or after identifying an object likely to be a microcalcification, a deconvolution can be performed to optimize the detection of the microcalcification. Accordingly, process 600 can include operations as described with respect to methods 400 and 500, such as receiving a reflection image (610), thresholding the image (620), removing skin voxels from the image (630), optionally removing outlier voxels (640), and performing morphological analysis (650). If there is a priori knowledge of the object, a deconvolution process (660) can be performed before the morphological analysis. Alternatively, the deconvolution process (660) can be performed once regions are identified as likely being microcalcifications (e.g., after the morphological analysis 650) to optimize the image. In another embodiment, the deconvolution process can be performed before spatial compounding of the individual views, or can be performed after spatial compounding and refraction correction (e.g., before or after processes relevant to 360/370 described with respect to FIG. 3). The deconvolution process can assist with identifying objects smaller than the resolution of the image since the signal for the microcalcification may bleed out into surrounding area/voxels and the deconvolution process can reduce the spread/remove the “bloom” effect.

Referring to FIG. 7, variable log compression can be applied. That is, instead of the reflection data being provided on a log scale or on a linear scale, ranges (i.e., subsets) of the data can be compressed. For example, lower intensity voxels may be compressed on a log scale, while the higher intensity voxels are maintained in linear scale. As shown in FIG. 7, the variable log compression can be applied after receiving the reflection image. In the example process 700, the reflection image is received (710), the variable log compression is applied to the data (720), the image is thresholded (730), skin is removed (740), outlier voxels are optionally removed (750), and morphological analysis is performed (760). In some cases, deconvolution or other optimizations may further be performed.

Some or all of process flow 300 described with respect to FIG. 3, process flow 400 described with respect to FIG. 4A, process flow 500 described with respect to FIG. 5A, process flow 600 described with respect to FIG. 6, and process flow 700 described with respect to FIG. 7 may be implemented in the form of computer-executable instructions, such as program modules, that are executed by one or more computers or other devices.

FIG. 8 shows an example computing system through which microcalcification detection may be carried out. In some implementations, the computing system may be embodied, at least in part, as a viewing station and/or PACS. In some implementations, the computing systems may embody, at least in part, the acquisition control system. Referring to FIG. 8, the system 800 can include a processor 810 and a storage system 820 in which instructions for calcification detection 830 may be stored. Examples of processor 810 include general purpose central processing units, application specific processors, and logic devices, as well as any other type of processing device, combinations, or variations thereof.

Storage system 820 includes any computer readable storage media readable by the processing system (e.g., processor 810) and capable of storing software, including instructions for calcification detection 830. Storage system 820 may include volatile and nonvolatile, removable and non-removable media implemented in any method or technology for storage of information, such as computer readable instructions, data structures, program modules, or other data. Examples of storage media include random access memory (RAM), read only memory (ROM), magnetic disks, optical disks, CDs, DVDs, flash memory, solid state memory, phase change memory, or any other suitable storage media. Certain implementations may involve either or both virtual memory and non-virtual memory. In no case do storage media consist of transitory propagated signals. In addition to storage media, in some implementations, storage system 820 may also include communication media over which software may be communicated internally or externally.

Storage system 820 may be implemented as a single storage device but may also be implemented across multiple storage devices or sub-systems co-located or distributed relative to each other. Storage system 820 may include additional elements, such as a controller, capable of communicating with processor 810.

The instructions for calcification detection 830 can direct the processor 810 to carry out any of the processes 400 such as described with respect to FIGS. 4A and 4B, processes 500 such as described with respect to FIGS. 5A and 5B, processes 600 such as described with respect to FIG. 6; and processes 700 such as described with respect to FIG. 7. In some cases, additional instructions may be stored, including those that can direct the processor 810 to carry out at least the image reconstruction processes of processes 300 (e.g., operations 360 and 370).

A database 840 storing at least the reflection imaging data from a QTUS system (or other system capable of appropriate resolution images) can be coupled to the system via wired or wireless connections.

Visual output can be provided via a display 850. Input/Output (I/O) devices (not shown) such as a keyboard, mouse, network card or other I/O device may also be included. It should be understood the any computing device implementing the described system may have additional features or functionality and is not limited to the configurations described herein.

In some embodiments, the machine/computer system can operate as a standalone device. In some embodiments, the machine/computer system may be connected (e.g., using a network) to other machines. In certain of such embodiments, the machine/computer system may operate in the capacity of a server or a client user machine in server-client user network environment, or as a peer machine in a peer-to-peer (or distributed) network environment.

The machine/computer system can be implemented as a desktop computer, a laptop computer, a tablet, a phone, a server, or any other machine capable of executing a set of instructions (sequential or otherwise) that specify actions to be taken by that machine, as well as multiple machines that individually or jointly execute a set (or multiple sets) of instructions to perform any one or more of the methods described herein.

The computer system can have hardware including one or more central processing units (CPUs) and/or digital signal processors (DSPs), memory, mass storage (e.g., hard drive, solid state drive), I/O devices (e.g., network interface, user input devices), and a display (e.g., touch screen, flat panel, liquid crystal display, solid state display). Elements of the computer system hardware can communicate with each other via a bus.

### Example—Sensitivity of QTUS to Detection of Microcalcifications

The example provided herein shows that QT reflection imaging can detect microcalcifications of size up to an order of magnitude smaller than the intrinsic resolution of the system. Imaging in custom fabricated phantoms show that the ability of QT imaging to detect calcium, as measured by contrast to noise ratio, is superior to x-ray mammography (XRM).

Custom phantoms with calcifications were fabricated. Agar phantoms provide comparable ultrasound characteristics to those of human tissue. FIG. 9A shows an agar phantom (liquid phase) with calcium particles to show a comparison with FIG. 9B, which shows a reflection image of the agar phantom. The agar phantom used in the image of FIG. 9B was formed as shown in FIGS. 10A-10C, where FIG. 10A shows the solid phase of the agar phantom of FIG. 9A; FIG. 10B shows the agar phantom in latex insert; and FIG. 10C shows the agar phantom with polymer coat.

Imaging performed by both QT and X-ray mammography (XRM) (performed on GE Senographe Essential) were compared and the respective contrast to noise ratios (CNR) as a function of size of calcium particles were calculated.

FIGS. 11A and 11B show an X-ray mammography image and a QT reflection image, respectively, of an agar phantom with calcium particles. In all instances, both QT imaging and XRM showed good visualization of calcium particles, as shown in FIGS. 11A and 11B. Results from imaging of phantoms show that QT images exhibit 23% higher CNR in detection calcium particles equal and greater than 500 μm, in comparison to XRM. For particles below 500 μm size, QT images exhibit 76% higher CNR.

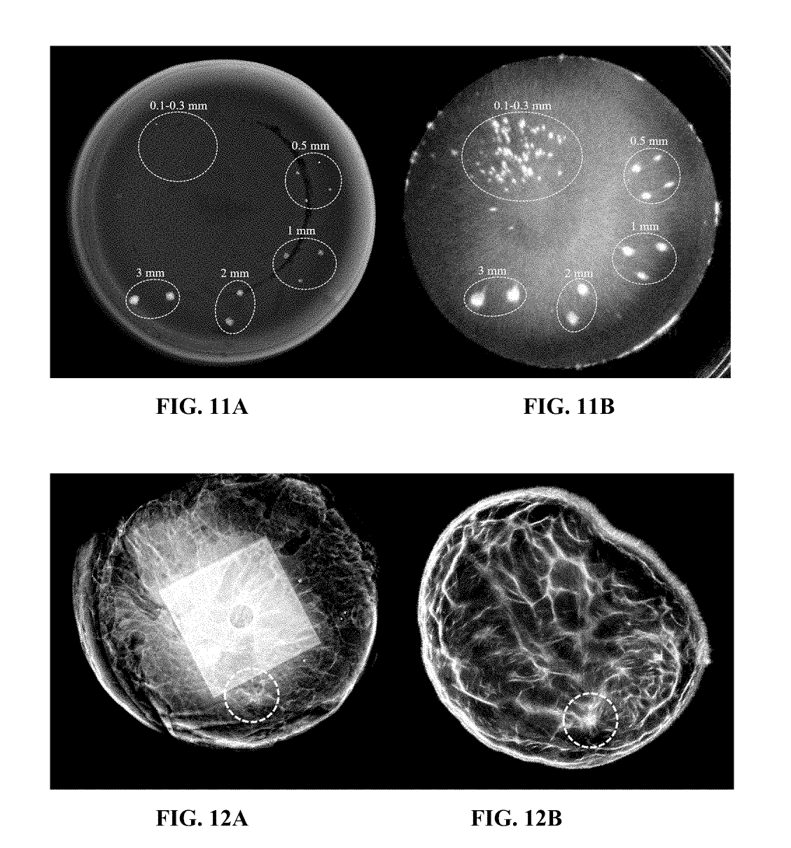

Also tested were cadaver breast tissue. FIG. 12A shows an X-ray mammography image of a cadaver breast tissue and FIG. 12B shows a QT reflection image of the cadaver breast tissue. As shown in FIGS. 12A and 12B, heterogeneous microcalcifications can be seen in the two images.

The functional block diagrams, operational scenarios and sequences, and flow diagrams provided in the Figures are representative of exemplary systems, environments, and methodologies for performing novel aspects of the disclosure. While, for purposes of simplicity of explanation, methods included herein may be in the form of a functional diagram, operational scenario or sequence, or flow diagram, and may be described as a series of acts, it is to be understood and appreciated that the methods are not limited by the order of acts, as some acts may, in accordance therewith, occur in a different order and/or concurrently with other acts from that shown and described herein. For example, those skilled in the art will understand and appreciate that a method could alternatively be represented as a series of interrelated states or events, such as in a state diagram. Moreover, not all acts illustrated in a methodology may be required for a novel implementation.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A system comprising:
a display;
a processor;
storage; and
instructions for performing calcification detection stored on the storage that when executed by the processor, direct the system to at least:
receive a reflection image of an object, the reflection image being a refraction-corrected reflection image generated from imaging data of a quantitative ultrasound imaging system;
perform a thresholding process on the reflection image;
remove skin voxels from the reflection image;
perform a morphology analysis using at least the reflection image to identify voxels likely to correspond to microcalcifications and discard voxels that are not likely to correspond to the microcalcifications; and
after the morphology analysis, provide a resulting image to the display, the resulting image indicating any microcalcifications in the reflection image using the voxels identified as likely to correspond to the microcalcifications.

2. The system of claim 1, wherein the instructions to perform the morphology analysis direct the system to:
identify voxels having an intensity higher than a threshold intensity as satisfying an intensity criterion; and
determine whether, for any arbitrary plane in the imaging data, there exists a set of adjacent voxels that satisfy the intensity criterion and a size criterion.

3. The system of claim 2, wherein the size criterion is a range between 5 adjacent voxels and 20 adjacent voxels.

4. The system of claim 1, further comprising instructions to:
perform, before the morphology analysis, image deconvolution using a prior knowledge of the object.

5. The system of claim 1, further comprising instructions to:
perform, after the morphology analysis, image deconvolution using regions having voxels identified as likely to correspond to the microcalcifications.

6. The system of claim 1, further comprising instructions to:
apply one or more image filters on the refraction-corrected reflection image to remove outlier voxels.

7. The system of claim 6, wherein the one or more image filters comprises a median filter.

8. The system of claim 6, wherein the one or more image filters comprises an image de-noising method.

9. The system of claim 1, further comprising instructions to:
perform image deconvolution on the reflection image.

10. The system of claim 1, further comprising instructions to:
apply a varying log compression to intensity values of the reflection image.

11. One or more computer-readable storage media having instructions stored thereon that when executed by a computing system, direct the computing system to at least:
perform a thresholding process on a reflection image;
remove skin voxels from the reflection image;
perform a morphology analysis to identify voxels likely to correspond to microcalcifications and discard voxels that are not likely to correspond to the microcalcifications, the morphology analysis directing the computing system to:
identify voxels having an intensity higher than a threshold intensity as satisfying an intensity criterion; and
determine whether, for any arbitrary plane in imaging data including the reflection image, there exists a set of adjacent voxels that satisfy the intensity criterion and a size criterion; and
after the morphology analysis, provide a resulting image to display, the resulting image indicating any microcalcifications in the reflection image using the voxels identified as likely to correspond to the microcalcifications.

12. The media of claim 11, further comprising instructions to generate the reflection image, the reflection image being a refraction-corrected reflection image generated from imaging data of a quantitative ultrasound imaging system.

13. The media of claim 12, further comprising instructions to:
perform image deconvolution before spatial compounding of individual views during generation of reflection images including the reflection image.

14. The media of claim 12, further comprising instructions to:
perform image deconvolution after spatial compounding of individual views and refraction-correction during generation of reflection images including the reflection image.

15. The media of claim 11, further comprising instructions to:
perform, before the morphology analysis, image deconvolution using a prior knowledge of an object in the reflection image.

16. The media of claim 11, further comprising instructions to:
perform, after the morphology analysis, image deconvolution using regions having voxels identified as likely to correspond to the microcalcifications.

17. A method of detecting microcalcifications, comprising:
receiving a refraction-corrected reflection image from imaging data of a quantitative ultrasound imaging system;
performing a thresholding process on the refraction-corrected reflection image;
removing skin voxels from the refraction-corrected reflection image; and
performing a morphology analysis to identify voxels likely to correspond to microcalcifications and discard voxels that are not likely to correspond to the microcalcifications; and
after performing the thresholding process, the removing of the skin voxels, and the morphology analysis on the refraction-corrected reflection image, providing a resulting image, the resulting image displaying any microcalcifications in the refraction-corrected reflection image.

18. The method of claim 17, further comprising:
applying one or more image filters on the refraction-corrected reflection image.

19. The method of claim 17, further comprising performing one or both of:
performing image deconvolution on uncompounded views and/or the refraction-corrected reflection image, the refraction-corrected reflection image being a compounded image;
applying a varying log compression to intensity values of the refraction-corrected reflection image.

20. The method of claim 17, wherein performing the morphology analysis comprises:
identifying voxels having an intensity higher than a threshold intensity as satisfying an intensity criterion; and
determining whether, for any arbitrary plane in the imaging data, there exists a set of adjacent voxels that satisfy the intensity criterion and a size criterion.
